(19) United States
(12) Patent Application Publication
(10) Pub. No.: US 2026/0325129 A1
(43) Pub. Date: Oct. 22, 2026

(54) SUPPORT DEVICE FOR AN IMPLANTABLE THREAD STRUCTURE, SYSTEM AND MEASURING METHOD HAVING SUCH A SUPPORT DEVICE, AND PRODUCTION METHOD FOR A SUPPORT DEVICE

(71) Applicant: KARL STORZ SE & Co. KG, Tuttlingen (DE)

(72) Inventors: Hannes BIEDERMANN, Tuttlingen (DE); Marian PFEFFER, Tuttlingen (DE)

(73) Assignee: KARL STORZ SE & Co. KG, Tuttlingen (DE)

(21) Appl. No.: 19/122,448

(22) PCT Filed: Oct. 18, 2023
(86) PCT No.: PCT/EP2023/078997

§ 371 Date: Apr. 18, 2025

(30) Foreign Application Priority Data
Oct. 19, 2022 (DE) 10 2022 127 583.3

(51) Int. Cl. A61B 17/06; A61B 17/04; A61B 90/00

(52) U.S. Cl. CPC A61B 17/06133; A61B 17/0401; A61B 90/06; A61B 2017/0404; A61B 2090/061

(57) ABSTRACT

The present disclosure relates to a support device for an implantable thread structure, wherein the support device has at least one support body for attaching the implantable thread structure, and at least one measuring device which is arranged on the support body and is designed to measure the implantable thread structure.

### CROSS-REFERENCE TO RELATED APPLICATIONS

This application is the U.S. national stage of PCT/EP2023/078997 filed on Oct. 18, 2023, which claims priority of German Patent Application No. DE 10 2022 127 583.3 filed on Oct. 19, 2022, the contents of which are incorporated herein.

### TECHNICAL FIELD

The present disclosure relates to a support device for an implantable thread structure and a system having such a support device and an implantable thread structure. Furthermore, the present disclosure relates to a production method for a support device and a measuring method for preconfiguring an implantable thread structure.

### BACKGROUND

Support devices for implantable thread structures are known from the prior art. The implantable thread structure is attached to the support device. The known support devices are intended exclusively for supporting the implantable thread configuration.

Implantable thread structures are required to fix a ligament or tendon graft in a desired position. For example, it could be a cruciate ligament graft that needs to be positioned and fixed accordingly. Implantable thread structures are known from the prior art, which have a holding element (“button”) and threads. The threads are connected to the holding element and can control the position of the holding element. The holding element can be pivoted (“flipped”) via the threads, for example, from an insertion position for guiding through a drill channel in a bone into a holding position in which the holding element rests against the bone and holds the thread structure and thus the graft. One of these known thread structures is disclosed, for example, in WO 2017/207027 A1.

These thread structures must be preconfigured and measured by the operating room staff before grafting. The preconfiguration of the thread structure leads to a reduction in friction between the thread structure threads and the graft, thus avoiding unnecessary stress on the graft. For example, if it is a cruciate ligament graft, the preconfiguration can also reduce the friction between the threads of the thread structure and the femoral condyle so that it is not damaged.

In order to set the required lengths and drilling depths for the drilling channel and the blind-hole and to configure the thread structure accordingly, the thread structure must be measured. A tendon board is necessary for preconfiguration and measurement of the thread structure. In addition, the distance (“flip distance”) by which the holding element of the thread structure must be guided out of the drill channel in order to pivot (“flip”) the holding element must be manually added to the desired drilling depth by the operating room staff or the surgeon himself.

### SUMMARY

Based upon this prior art, it is an object of the present disclosure to simplify the preconfiguration and measurement of the thread structure.

This object is achieved with a support device for an implantable thread structure according to claim 1, a system according to claim 16, and a measuring method according to claim 21.

The support device has at least one support body for attaching the implantable thread structure and at least one measuring device which is arranged on the support body and is designed to measure the implantable thread structure.

With the integrated measuring device of the support device, the thread structure together with the graft can be measured directly on the support device. This eliminates the need for a tendon board in the operating room. The handling and preparation of the thread structure including the graft are considerably simplified with the support device according to the disclosure, and the susceptibility to errors is reduced.

The at least one measuring device may have a first measuring scale and a second measuring scale. The first measuring scale and the second measuring scale can be arranged in succession in the longitudinal direction of the support device. The first measuring scale and the second measuring scale can be provided for measuring a first length (first measuring scale) and a second length (second measuring scale). The first measuring scale and the second measuring scale can indicate millimeters as the unit of length.

The first measuring scale and the second measuring scale may be at a predetermined distance from one another in the longitudinal direction of the support device. With this predetermined distance, the lengths measured with the first measuring scale and the second measuring scale can be increased by a predetermined length amount. These measured lengths, which are increased by the length of the predetermined distance, can prevent damage to the soft tissue, which can occur, for example, if excessive pressure is exerted on the soft tissue surrounding the drill hole via the holding element in the holding position described above.

The at least one support device may have a first positioning device and a second positioning device for positioning the implantable thread structure on the support body. The first positioning device and the second positioning device can be offset from one another in the longitudinal direction of the support device.

The first positioning device can be arranged at a predetermined position relative to the first measuring scale in the longitudinal direction of the support device. The first positioning device can be arranged at the zero value of the first measuring scale. The at least one second positioning device can be arranged at a predetermined position relative to the second measuring scale. The second positioning device can be arranged at the zero value of the second measuring scale.

The second measuring scale and the second positioning device can be arranged on the support device in such a way that a predetermined distance is established between the second measuring scale and the second positioning device in the longitudinal direction of the support device. The at least one predetermined distance may be determined based upon the shape and/or dimensions of a retaining element of the thread structure. The predetermined distance may correspond to the distance explained above that is required to pivot (“flip”) the holding element into a holding position on the bone. This distance can also be referred to as the “flip distance.” The “flip distance” thereby no longer needs to be added to the second measured length by the operating room staff, which can prevent errors.

The first positioning device may have at least two positioning elements. The positioning elements can be designed to deflect the implantable thread structure. The positioning elements can be elements around which the thread structure can be placed. The positioning elements can be designed such that the thread structure between the positioning elements runs substantially orthogonal to the longitudinal axis of the support device.

The second positioning device may have at least one positioning projection. The positioning projection may be configured to position the holding element of the thread structure in a predetermined position on the support device. The positioning projection may have a front side and a rear side. The positioning projection can be designed such that the holding element of the thread structure can be supported on the rear side of the positioning projection. The rear side of the positioning projection may face away from the first positioning device.

The first positioning device can define a connection position for the graft with the implantable thread structure. The second positioning device can adjust the position of the graft relative to the holding element of the thread structure. When the implantable thread structure is located on the first positioning device and the second positioning device, i.e., in the starting position on the support device, the distance between the graft attached to the thread structure and the holding element can correspond to the distance in the longitudinal direction between the first positioning device and the second positioning device.

The positioning projection may have an opening for passing the threads of the thread structure through. The positioning projection can be slotted. The slot of the positioning projection can be used to insert the threads of the thread structure into the opening of the positioning projection. This can facilitate the attachment of the thread structure to the support device and also the positioning of the holding element on the positioning projection.

The support device may have at least one receiving opening. The at least one receiving opening can be arranged between the at least two positioning elements of the first positioning device. A graft can be inserted into the receiving opening for connection to the thread structure. The receiving opening can extend from the first positioning device in the direction of the second positioning device into the support body. The receiving opening can widen in the transverse direction along the longitudinal direction of the support device. This allows an elasticity of the support device to be adjusted in the region of the receiving opening, which can facilitate the release of the thread structure from the first positioning device. The positioning elements of the first positioning device can be designed such that the thread structure spans the receiving opening.

A graft can be placed around the portion of the thread structure that extends between the two positioning elements, to connect to the thread structure through the receiving opening. The portion of the thread structure extending between the two positioning elements may form a connection portion for connecting the graft to the thread structure.

The support device may have at least one receiving area for threads of the thread structure. The receiving area can be formed on the rear side of the support body.

The support device may have at least one tab. The at least one tab can be displaceable relative to the support body and connectable thereto to form the receiving area.

The tab can be folded along one edge of the support body. This edge can be called a “folding edge.” The tabs can be folded to the rear side of the support body and connected to the support body with their free end. This allows a pocket to be formed on the rear side of the support body for the threads of the thread structure.

The support body may have at least one slot or insertion window into which the tab can be inserted with its free end. The tab can be designed with two tab arms. The free ends of the tab arms can be inserted into the slot or the insertion window.

At least one of the tab arms may have an opening to which the ends of the threads can be fixed. The ends of the threads can be fixed to this opening when the receiving area is open and/or closed.

The support device may have at least one passage opening for the threads of the thread structure. The threads of the thread structure can be guided from the front side of the support body to its rear side through the passage opening. The passage opening can be formed at the transition between the support body and the tab. The threads can be guided into or out of the receiving area via the passage opening.

At least the at least one measuring device can be formed integrally with the at least one support body. The tab can also be formed in one piece with the support body. The entire support device can be formed in one piece.

The support device can be made of plastic.

The system according to the disclosure has a support device of the type described above and a thread structure attached to the support body of the support device.

The system can be treated as a unit. The system with the support device and the attached thread structure can be sold as a unit and used in the operating room. No additional components or equipment are required for preconfiguration, since the support device is designed not only to support the thread structure, but also to measure and preconfigure the thread structure with the graft.

The first positioning device and the second positioning device can position the implantable thread structure in a predetermined position on the support body. A holding element of the thread structure is supported on the second positioning device, and threads of the thread structure are received in the receiving area of the support device.

A production method for producing a support device for an implantable thread structure comprises the following steps:
providing a plate-shaped main body, and
producing the support body of the support device from the plate-shaped main body, wherein the first positioning device, the second positioning device, the at least one tab, and the at least one measuring device are formed on the support body in one operation.

The support device can be produced quickly and easily in just a few method steps. Since the support device is produced from a main body using the production method, the support device has a simple structure without loose individual parts.

The production method may include the following step:
applying the first measuring scale and the second measuring scale to the support body.

The first and second measuring scales can be printed or stamped on the support body. Alternatively, the first and second measuring scales can be embossed into the support body.

The support body of the support device can be produced by means of a punching process. The entire support device can be produced using a punching process. This means that the support body and also the tab connected to the support body can be punched out of a main body. By folding and/or unfolding, the individual elements of the support device can be brought into their predetermined position and/or placement. Furthermore, elements such as the edge of the tab can also be embossed into the main body during the punching process.

A measuring method for preconfiguring an implantable thread structure with the system described above comprises the following steps:
fastening a graft to the thread structure in the region of the receiving opening of the support device,
determining a first length using the first measuring scale;
adjusting a second length by actuating the thread structure, wherein a portion of the thread structure together with the graft is displaced relative to the second positioning device in accordance with the second measuring scale and the determined first length.

The first positioning device can define a connection position for the graft with the implantable thread structure. The connection between the graft and the thread structure can be made in the region of the receiving opening on the first positioning device. The first positioning device can adjust a course of the thread structure such that a connection portion of the thread structure is formed for connection to the graft. The holding element of the thread structure can be located on the second fastening device. In the initial position of the thread structure on the support device, the distance between the graft attached to the thread structure and the holding element can correspond to the distance in the longitudinal direction between the first positioning device and the second positioning device.

In the initial position of the thread structure and the attached graft, the first length can be determined using the first measuring scale. The determined first length can be removed from the graft with reference to the first measuring scale. After removing the first length of the graft, the thread structure can be removed from the first positioning device.

To adjust the second length, the thread structure is actuated. This changes the position of the graft relative to the second positioning device, on which the holding element of the thread structure is located. The movement of the graft due to the actuation of the thread structure can occur substantially in the longitudinal direction of the support device. If the second length could be set according to the second measuring scale, the preconfiguration of the thread structure with the graft and the measurement are finished. For the preconfiguration and measurement, no additional components or equipment such as a tendon board were required besides the support device.

The measuring method may include the following step:
opening the receiving area of the support device in order to actuate the thread structure to adjust the second length.

To open the receiving area, the free end of the tab of the support device can be detached from the support body. This allows the flap to be opened and the threads to be released to actuate the thread structure.

The disclosure is explained below using figures by way of example. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using numerical words, such as first, second, third object, etc., these are used to name and/or assign objects. Accordingly, for example, a first object and a third object may be included, but not a second object. However, a number and/or sequence of objects could also be derived using numerical words.

### BRIEF DESCRIPTION OF DRAWINGS

FIG. 1 is a perspectival view of a support device;

FIG. 2 is a perspectival view of the support device with closed receiving area;

FIG. 3 is a perspectival view of a system with the support device according to FIGS. 1 and 2 and a thread structure attached thereto;

FIG. 4 is a perspectival view of the system according to FIG. 3 and a graft attached to the thread structure; and

FIG. 5 is a perspectival view of the system according to FIG. 3 with a graft attached and with the receiving area opened.

### DETAILED DESCRIPTION OF PREFERRED EMBODIMENTS

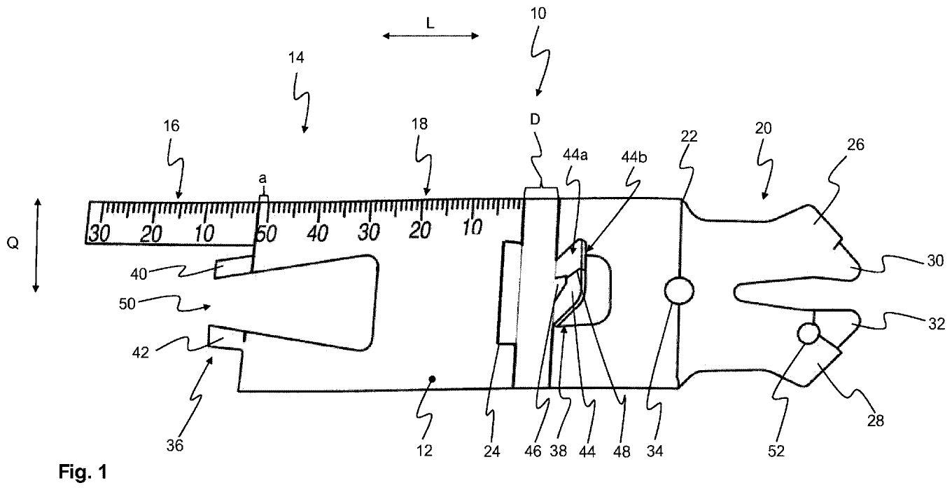

FIG. 1 shows a perspectival view of a support device 10 for an implantable thread structure (not shown in FIG. 1). The support device 10 has a support body 12 to which the thread structure can be attached. A measuring device 14 is arranged on the support body 12. The measuring device 14 has a first measuring scale 16 and a second measuring scale 18. The first measuring scale 16 and the second measuring scale 18 are arranged in succession in the longitudinal direction L of the support device 10. The first measuring scale 16 and the second measuring scale 18 are arranged on the support body 12 at a distance from one another in the longitudinal direction L of the support device 10. A predetermined distance a is thus established between the first measuring scale 16 and the second measuring scale 18. The first measuring scale 16 and the second measuring scale 18 extend along an edge of the support body 12 running in the longitudinal direction L. The first measuring scale 16 is arranged on a strip-shaped portion of the support body 12.

The support device 10 has a tab 20 which is connected to the support body 12. The tab 20 can be displaced relative to the support body 12 to form a receiving area for threads of the thread structure. The tab 20 can be folded along a folding edge 22 towards the rear side of the support body 12. The folding edge 22 may be an embossed edge to facilitate folding of the flap 20. To form the receiving area, the tab 20 can be folded over and connected with its free end to the support body 12. The support body 12 has an insertion window 24 for inserting the free end of the tab 20.

The tab 20 has two tab arms 26, 28 which extend away from the support body 12 in the longitudinal direction L of the support device 10. By folding the tab 20 at the folding edge 22, the free ends 30, 32 of the tab arms 26, 28 can be inserted into the insertion window 24 on the support body 12 in order to form the receiving area on the rear side of the support body 12. The free ends 30, 32 of the tab arms 26, 28 are inserted from the rear side of the support body 12 into the insertion window 24 so that they are fixed to the support body 12.

The support device 10 further has a passage opening 34. The passage opening 34 is formed partly on the support body 12 and partly on the tab 20. The passage opening 34 divides the folding edge 22. The passage opening 34 is formed approximately in the middle of the folding edge 22. The threads of the thread structure can run through the passage opening 34.

The support device 10 has a first positioning device 36 and a second positioning device 38, which are designed to position the implantable thread structure on the support body 12. The first positioning device 36 and the second positioning device 38 are offset from one another in the longitudinal direction of the support device 10. The first positioning device 36 and the second positioning device 38 are thus at a predetermined distance from each other in the longitudinal direction of the support device 10.

The first positioning device 36 has two positioning elements 40 and 42. The positioning elements 40, 42 are offset from one another in the transverse direction Q. The positioning elements 40 and 42 are designed such that they can guide and redirect threads of the implantable thread structure. The positioning elements 40, 42 can be designed as positioning lugs which protrude from the support body 12 in the longitudinal direction L of the support device 10. The positioning elements 40, 42 provide a predetermined course for the threads of the thread structure in the region of the first positioning device 36.

The second positioning device 38 has a positioning projection 44. An opening 46 extends through the positioning projection 44, and the threads of the thread structure can be guided through said opening. The positioning projection 44 can be slotted. The threads can be inserted into or removed from the opening 46 through the slot 48. The positioning projection 44 has a front side 44a and a rear side 44b. A holding element (not shown in FIG. 1) of the thread structure can be supported on the rear side 44b of the positioning projection 44.

The support device 10 has a receiving opening 50. The receiving opening 50 is formed on the support body 12. A graft (not shown) can be passed through the receiving opening 50 in order to be connected to the thread structure. The receiving opening 50 extends between the two positioning elements 40 and 42 into the support body 12. The receiving opening 50 widens along the longitudinal direction L of the support device 10 in the transverse direction Q. Due to the widening receiving opening 50, an elasticity of the support device 10 can be achieved in the region of the receiving opening 50 and can facilitate a release of the thread structure from the first positioning device 36.

The first positioning device 36 is arranged at a predetermined position relative to the first measuring scale 16. The first positioning device 36 establishes a first predetermined position for connecting the thread structure to a graft. The first positioning device 36 positions the thread structure on the support device 10 such that a connection portion of the thread structure for connection to the graft is positioned at the zero value of the first measuring scale 16.

The second positioning device 38 is arranged at a predetermined position relative to the second measuring scale 18. The second positioning device 38 and the second measuring scale 18 are arranged on the support device 10 such that a predetermined distance D is established between the second measuring scale 18 and the second positioning device 38. The predetermined distance D is set between the positioning projection 44 and the zero value of the second measuring scale 18. The numerical values of the second measuring scale 18 increase towards the first measuring scale 16.

The support device 10 is formed in one piece. The support device 10 can be produced from a plate-shaped main body by means of a punching process. The elements described above can be unfolded or folded into their predetermined position and placement if necessary.

The tab arm 28 of the tab 20 has an opening 52. The opening 52 can serve to fix the ends of the threads of the thread structure to the tab 20 or to the support device 10.

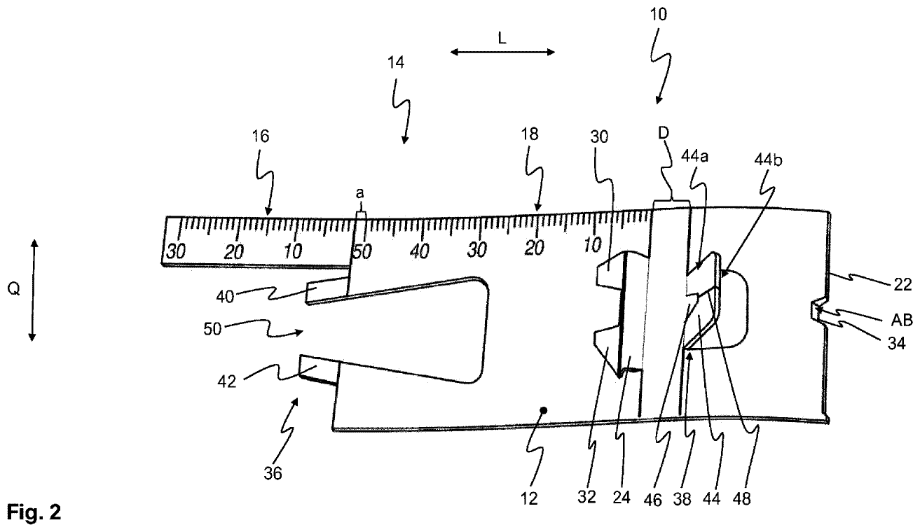

FIG. 2 shows a perspectival view of the support device 10 in which the tab 20 (see FIG. 1) has been folded towards the rear side of the support body 12. The free ends 30, 32 of the tab arms 26, 28 have been inserted into the insertion window 24 from the rear side of the support body 12. The free ends 30, 32 protrude through the insertion window 24 on the front side of the support body 12. This forms the receiving area AB for the threads of the thread structure on the rear side of the support body 12. The front side of the support body 12 and the support device 10 as a whole is defined by the first measuring scale 16 and the second measuring scale 18, i.e., the first measuring scale 16 and the second measuring scale 18 are located on the front side of the support body 12.

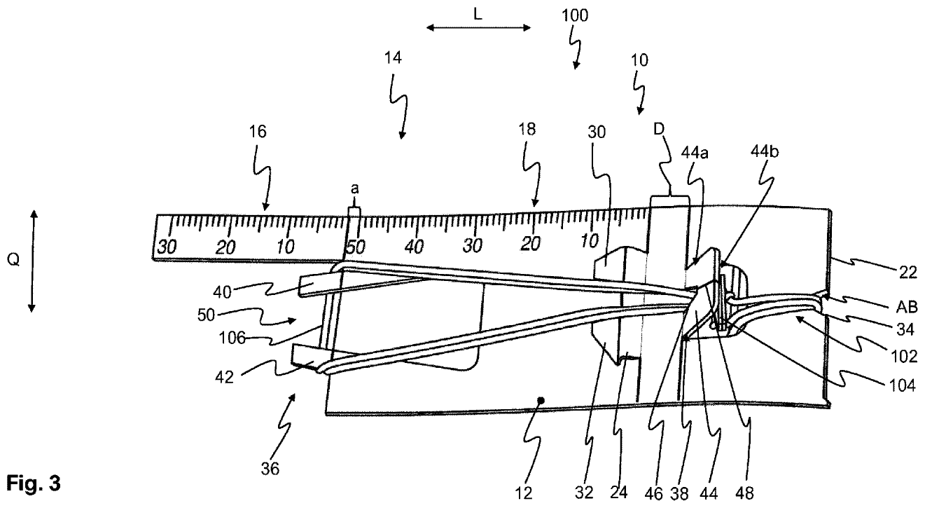

FIG. 3 shows a perspectival view of a system 100 comprising the support device 10 and a thread structure 102. The system 100 forms a unit. This unit may be packaged and distributed in the state shown in FIG. 3. In the operating room, this unit can be unpacked and is ready for use without additional components for connection to a graft, as well as for preconfiguration and measurement.

The thread structure 102 is attached to the support body 12. The first positioning device 36 and the second positioning device 38 position the thread structure 102 in a predetermined position on the support body 12. The thread structure 102 is placed with its loop-shaped portion around the two positioning elements 40, 42 of the first positioning device 36. Starting from the first positioning device 36, the thread structure 102 extends in the direction of the second positioning device 38. The thread structure 102 is inserted into the opening 46 of the positioning projection 44 via the slot 48 in the positioning projection 44. In the state shown in FIG. 3, the thread structure 102 extends through the opening 46 in the positioning projection 44.

The thread structure 102 has a holding element 104, which is also referred to as a “button.” The holding element 104 is supported on the rear side 44b of the positioning projection 44. The thread structure 102 extends through the passage opening 34 into the receiving area AB on the rear side of the support body 12. On the front side of the support body 12 in FIG. 3, the ends 30, 32 of the tab arms 26, 28 are shown, which protrude through the insertion window 24. In other words, the thread structure 102 is looped around the positioning elements 40, 42 and guided through the opening 46 of the positioning projection 44, so that the holding element 104 of the thread structure 102 can be supported on the positioning projection 44. The threads of the thread structure 102 are guided through the passage opening 34 into the receiving area AB.

The positioning elements 40, 42 of the first positioning device 36 deflect the thread structure 102 such that a portion 106 of the thread structure 102 extends between the positioning elements 40, 42 and spans the receiving opening 50 in this region. The portion 106 of the thread structure 102 forms a connection portion to which a graft, not shown in FIG. 3, can be attached. The graft can be placed through the receiving opening around the connection portion 106 of the thread structure and connected to the thread structure 102. The thread structure 102 runs in the portion 106 substantially orthogonally to the longitudinal direction L of the support device 10.

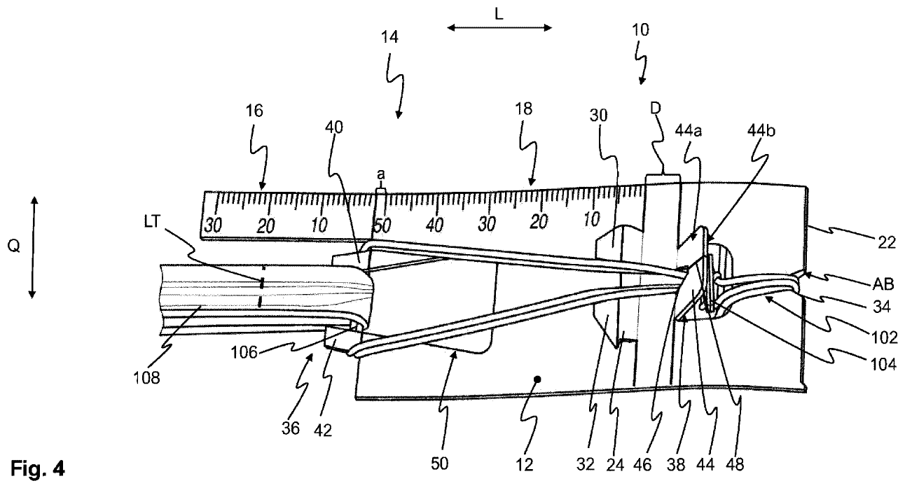

FIG. 4 shows a perspectival view of the system 100 with a graft 108 connected to the thread structure 102. The graft 108 has been placed through the receiving opening 50 around the connection portion 106 of the thread structure 102 and connected to the thread structure 102. The thread structure 102 is guided around the positioning elements 40, 42 in such a way that the thread structure 102 runs along the rear side of the positioning elements 40, 42.

In the state of the system 100 and the graft 108 shown in FIG. 4, a first length can be determined with the first measuring scale 16. The first length can, for example, be the length or drilling depth of a femoral blind-hole. The determined first length is removed from the graft 108. This is shown in FIG. 4 by way of example by the dashed line LT. The determined first length LT can, for example, be 20 mm.

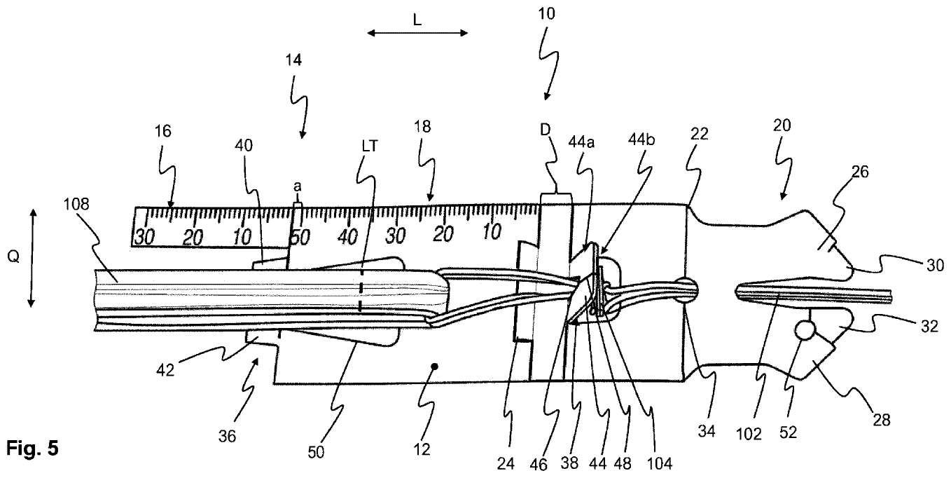

Following the determination of the first length, the thread structure 102 is released from the first positioning device 36 and thus from the positioning elements 40, 42. By actuating the thread structure 102, a relative movement of the loop-shaped portion of the thread structure 102 together with the graft relative to the positioning projection 44 of the second positioning device 38 is carried out. The holding element 104 of the thread structure 102 is supported on the rear side 44b of the positioning projection 44 during this relative movement. Now, a second length can be determined using the second measuring scale 18. This state is shown in FIG. 5.

In order to be able to actuate the thread structure 102, the tab 20 has been detached from the support body 12, and the receiving area AB opened. By opening the receiving area AB, the threads of the thread structure 102 can be actuated, whereby the relative position of the graft 108 relative to the positioning projection 44 of the second positioning device 38, and thus to the holding element 104 of the thread structure 102, can be changed. This allows the second length to be set relative to the second measuring scale 18.

If the marking LT for the first length is at the desired position on the second measuring scale 18, the actuation of the thread structure 102 is terminated. The second length can, for example, indicate the length or drilling depth of the femoral canal. The second length can, for example, be 35 mm.

Due to the predetermined distance D between the second positioning device 38 and the zero value of the second measuring scale 18, no further length amount needs to be added to the second set length, since the predetermined distance D corresponds to the distance required to pivot or “flip” the holding element 104. The holding element 104 is guided through the femoral canal in an insertion position and must then be pivoted into a holding position suitable for placement on the bone. In order to have sufficient space for pivoting (“flipping”) the holding element 104, the holding element 104 must be moved a predetermined distance out of the femoral canal. The additional distance D corresponds to the flip distance by which the holding element 104 must be moved out of the femoral canal in order to be able to pivot. The addition of the flip distance to the second length by the operating room staff or the surgeon, as described above, is therefore no longer necessary. The support device 10 can thus prevent errors.

1. A support device for an implantable thread structure, wherein the support device has:
at least one support body for attaching the implantable thread structure, and
at least one measuring device which is arranged on the support body and is designed to measure the implantable thread structure.

2. The support device according to claim 1, herein the at least one measuring device has a first measuring scale and a second measuring scale.

3. The support device according to claim 2, herein the first measuring scale and the second measuring scale are arranged in succession in the longitudinal direction of the support device.

4. The support device according to claim 2, herein the first measuring scale and the second measuring scale are at a predetermined distance from one another in the longitudinal direction of the support device.

5. The support device according to claim 1, wherein the at least one support device has a first positioning device and a second positioning device for positioning the implantable thread structure on the support body, wherein the first positioning device and the second positioning device are offset from one another in the longitudinal direction of the support device.

6. The support device according to claim 5, wherein the first positioning device relative to the first measuring scale and the at least one second positioning device relative to the second measuring scale are arranged at predetermined positions in the longitudinal direction of the support device.

7. The support device according to claim 5, wherein the second measuring scale and the second positioning device are arranged on the support device in such a way that a predetermined distance is established between the second measuring scale and the second positioning device in the longitudinal direction of the support device.

8. The support device according to claim 5, wherein the first positioning device has at least two positioning elements which are designed to deflect the implantable thread structure.

9. The support device according to claim 5, wherein the second positioning device has at least one positioning projection which is designed to position a holding element of the thread structure in a predetermined position on the support device.

10. The support device according to claim 8, wherein the support device has at least one receiving opening which is arranged between the at least two positioning elements of the first positioning device.

11. The support device according to claim 1, wherein the support device has at least one receiving area for threads of the thread structure .

12. The support device according to claim 11, wherein the support device has at least one tab which can be displaced relative to the support body and connected thereto to form the receiving area.

13. The support device according to claim 1, wherein the support device has at least one passage opening for the threads of the thread structure.

14. The support device according to claim 1, wherein at least the at least one measuring device is formed integrally with the at least one support body.

15. The support device according to claim 1, wherein the support body is made of plastic.

16. A system with a support device according to claim 1 and a thread structure attached to the support body of the support device.

17. The system according to claim 16, wherein the first positioning device and the second positioning device position the implantable thread structure in a predetermined position on the support body, wherein a holding element of the thread structure is supported on the second positioning device, and threads of the thread structure are received in the receiving area of the support device.

18. (canceled)

19. (canceled)

20. (canceled)

21. A measuring method for preconfiguring an implantable thread structure with a system according to claim 16, wherein the measuring method comprises the following steps:
fastening a graft to the thread structure in the region of the receiving opening of the support device,
determining a first length using the first measuring scale;
adjusting a second length by actuating the thread structure, wherein a portion of the thread structure together with the graft is displaced relative to the second positioning device in accordance with the second measuring scale and the determined first length.

22. The measuring method according to claim 21, wherein the measuring method comprises the following step:
opening the receiving area of the support device in order to actuate the thread structure to adjust the second length.
